(19) United States
(12) Patent Application Publication
(10) Pub. No.: US 2023/0138074 A1
(43) Pub. Date: May 4, 2023

(54) DEVICE AND METHOD FOR CONTROLLING A LIGHTING AND SANITIZING SYSTEM

(71) Applicant: ARTEMIDE S.P.A., Milano (IT)

(72) Inventors: Carlotta Francesca Isolina Maria DE BEVILACQUA, Milano (IT); Andrea GALLUCCI, Milano (IT)

(21) Appl. No.: 17/974,244

(22) Filed: Oct. 26, 2022

(30) Foreign Application Priority Data
Nov. 4, 2021 (IT) 102021000028142

(51) Int. Cl. H05B 47/25; H05B 47/115; H05B 47/19; A61L 2/24; A61L 2/10

(52) U.S. Cl. CPC H05B 47/25; H05B 47/115; H05B 47/19; A61L 2/24; A61L 2/10; H05B 47/16

(57) ABSTRACT

A control device for controlling a lighting system with sanitizing function comprises a printed circuit board connectable to at least one lamp of the lighting system provided with at least one sanitizing source; at least one presence sensor connected to the board by a communication system, for example a wireless communication system; an amperometric sensor connected to the sanitizing source to measure the current in the sanitizing source; the board is configured so as to disable the sanitizing source or to reduce the power supply to the sanitizing source below a threshold value when the presence sensor detects the presence of users; or when a malfunction is detected in the communication system and the amperometric sensor detects in the sanitizing source a current greater than the threshold value.

### CROSS-REFERENCE TO RELATED APPLICATIONS

This patent application claims priority from Italian Patent Application no. 102021000028142 filed on Nov. 4, 2021, the entire disclosure of which is incorporated herein by reference.

### TECHNICAL FIELD

The present invention relates to a device and method for controlling a lighting system which integrates lighting and sanitizing (disinfectant, antibacterial, etc.) functions.

### BACKGROUND

The use of UV lamps (in particular, UV lamps emitting in the UV-C band) with sanitizing function is well known, UV light being capable of deactivating or eliminating pathogenic microorganisms.

On the other hand, UV-C light is not suitable for lighting environments and also has potentially dangerous effects on humans too, if not properly controlled.

It is also known that certain light radiations in the visible range, particularly in the violet range, can have disinfectant effects. Lighting devices are therefore known which include a light source emitting specific-wavelength violet light with disinfectant function combined with a light source emitting a complementary light resulting in white light.

In general, well-known lighting devices that are also equipped with UV-C sources and/or violet light sources with sanitizing function are not fully satisfactory.

In particular, the problem arises of a safe control of potentially hazardous emissions for users. In fact, not only UV radiation, but also violet light having a wavelength suitable for the sanitizing function can be harmful, especially if users are exposed for long periods and/or at high intensity.

### SUMMARY

The object of the present invention is to overcome the drawbacks of the prior art, in particular by providing a device and a method for controlling a lighting and sanitizing system which is simple and reliable and fully efficient and safe.

The present invention therefore relates to a device and a method for controlling a lighting and sanitizing system as defined in appended claims 1 and 10, respectively.

The invention provides a device and method for controlling a lighting and sanitizing system, which is simple and reliable, and fully efficient and safe. In particular, the control device and method of the invention ensure that the sanitizing source, potentially dangerous to users, is only activated under full safety conditions.

### BRIEF DESCRIPTION OF THE DRAWINGS

Further features and advantages of the present invention will be apparent from the description of the following non-limiting embodiment with reference to the figures of the accompanying drawings, wherein:

FIG. 1 is a schematic view of a lighting and sanitizing system equipped with a control device according to the invention;

FIG. 2 is a schematic view of the control device of the invention.

### DESCRIPTION OF EMBODIMENTS

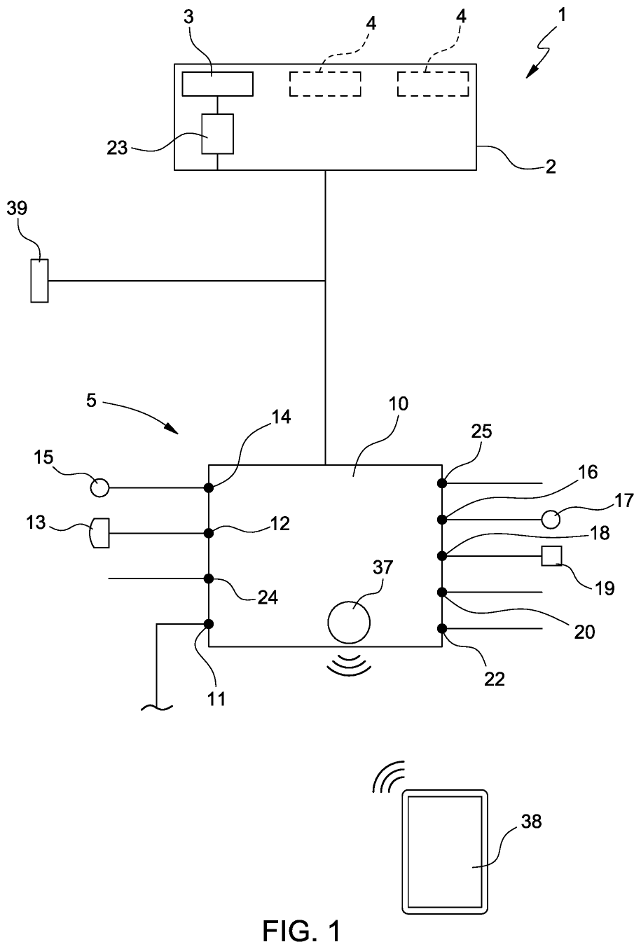

The numeral 1 in FIG. 1 shows, as a whole, a lighting system with lighting and sanitizing functions, for example, operating in an environment (one or more rooms).

The lighting system 1 comprises at least one lamp 2 including at least one sanitizing source 3, which emits radiation capable of performing a sanitizing function, such as for example UV radiation, in particular UV-C, or violet light having a wavelength between 380 and 430 nm.

The lamp 2 may include one or more sanitizing sources 3, even different ones (e.g., one or more UV sources and one or more violet light sources); optionally, the lamp 2 may also include one or more visible light sources 4 emitting white light, alone and/or in combination with respective violet light sources.

The lighting system 1 may comprise a plurality of lamps 2, even different ones, i.e., equipped with different sanitizing sources 3 and/or different visible light sources 4.

For example (but not necessarily), the lighting system 1 comprises one or more lamps of the type described in Italian Patent Application no. 102020000010399.

For the sake of simplicity, reference will be made hereinafter to a lighting system 1 with a single lamp 2 equipped with one sanitizing source 3, but it is understood that the invention can also be implemented with several lamps 2 and/or several sanitizing sources 3.

The lighting system 1 comprises a control device 5 connected to the lamp 2 and controlling the lamp 2, and in particular the sanitizing source 3, in order to avoid health risks to users who may be exposed to the emission from the sanitizing source 3.

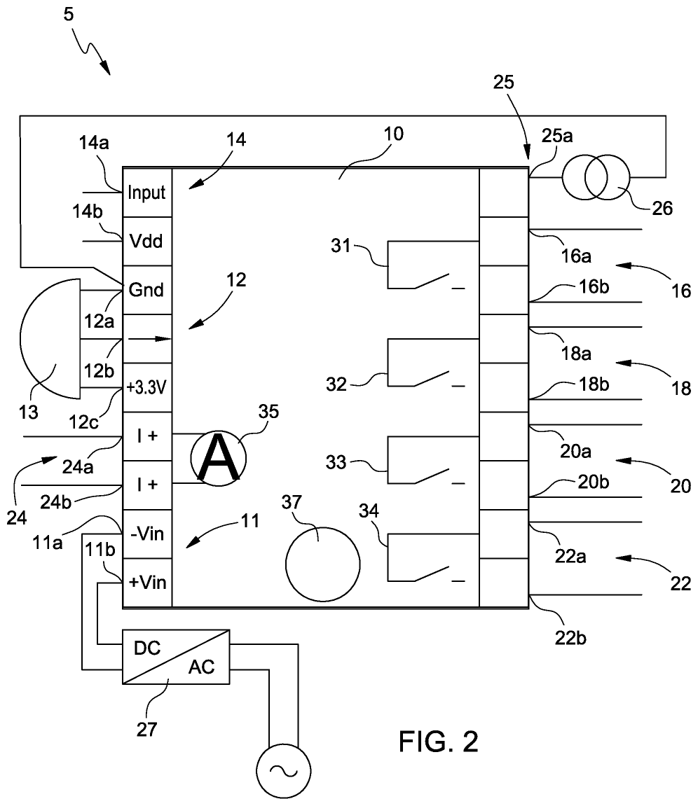

Also referring to FIG. 2, the control device 5 comprises, in particular, a printed circuit board 10, comprising:

a supply input 11 for connection to a power supply, for example a 12-48 V direct current power supply;

a sensor input 12 connected to a presence sensor 13, for example a PIR sensor (passive infrared sensor);

an outer sensor input 14 for connection to further outer sensors 15, e.g., one or more further presence sensors and/or door opening sensors and/or a consent sensor (which can be activated, for example, by means of a key or an unlock code);

a first relay contact output 16 for connection to a visual signalling LED 17;

a second relay contact output 18 for connection to a sound signal 19 (a buzzer);

a third relay contact output 20 for sanitization in progress;

a fourth relay contact output 22 for connection in series to a driver 23 of the sanitizing source 3;

a current measure input 24 for measuring the current in the sanitizing source 3;

a transformer input 25 for connection to an amperometric transformer 26 for measuring the primary current in the driver 23 of the sanitizing source 3.

The supply input 11 comprises, for example, a pair of supply terminals 11a, 11b connected to a power supply 27.

The sensor input 12 comprises three terminals 12a, 12b, 12c, including an earth terminal 12a, which are connected to the presence sensor 13. The presence sensor 13 is placed in the room where the lighting system 1 is located to detect the presence of people in this room.

The outer sensor input 14 comprises a pair of terminals 14a, 14b connected to the sensors 15, in particular one or more door opening sensors acting on respective doors of the room in which the lighting system 1 operates, to detect the opening of these doors.

The first output 16 is the output of a first relay 31, in particular a low voltage (max 50V, 100 mA) static relay, and comprises a pair of terminals 16a, 16b connected to the visual signalling LED 17, which is placed in an appropriate position to send a visual signal (steady or flashing light) to users.

The second output 18 is the output of a second relay 32, in particular a low voltage (max 50V, 100 mA) static relay, and comprises a pair of terminals 18a, 18b connected to the sound signal 19, which is placed in an appropriate position to send a sound signal to users.

The third output 20 is the output of a third relay 33 to signal sanitization in progress (e.g., max 250V, 3 A), and comprises a pair of terminals 20a, 20b to indicate that the sanitizing source 3 is switched on and sanitization is in progress.

The fourth output 22 is the output of a fourth relay 34 (e.g., max 250V, 3 A) connected in series to the driver 23 of the sanitizing source 3 and comprises a pair of terminals 20a, 20b.

The current measure input 24 comprises a pair of terminals 24a, 24b connected to the sanitizing source 3 to measure, via an amperometric sensor 35, the current to the lamp load (secondary driver), i.e., the current in the sanitizing source 3 on the secondary side of the respective driver 23.

The transformer input 25 comprises a terminal 25a connected to the transformer 26 for measuring the primary current in the driver 23 of the sanitizing source 3. The other wire of the transformer 26 is inserted in the earth terminal 12a.

The control device 5 is then provided with a communication system 37, preferably a wireless communication system, for example Bluetooth, to communicate, other than with the presence sensor 13 and the other sensors 15, with a remote device 38, for example, a smartphone or other similar device loaded with a dedicated application (app) providing a user interface with the control device 5.

The operation of the control device 5 and thus of the lighting system 1, for implementing the method according to the invention, is as follows.

Via the app (and therefore via the device 38) the user can select various functions, in particular:
safety chain operation test: the app activates a command setting the control device 5 into test mode. In this mode, if the presence sensor 13 and/or one of the other sensors 15 (e.g., a door opening sensor) detects a presence or the opening of a door:

1) the first output 16 is activated and the visual signalling LED 17 switches on; in this way, a visual test is carried out for the correct functioning of the safety chain, which activates instantaneously if users are present; and

2) the second output 18 is activated and the sound signal 19 switches on; in this way, a sound test is carried out for the correct functioning of the safety chain, which activates instantaneously if users are present;
activation of sanitization: either via the app or via a switch 39 (e.g., a b11-type switch), the sanitizing source 3 (or the entire lamp 2) is switched on; the control device 5 provides the consent to switch on the sanitizing source 3.

In particular, the app (via the device 38) or the switch 39 sends a command to the control device 5; after a preset exit time (which can be set through the app to allow exit from the room), the control device 5 sends an indirect command to the sanitizing source 3. The third relay 33 closes to indicate that sanitization is on.

During the exit time, the first relay 31 (for the visual signalling LED 17) and the second relay 32 (for the sound signal 19) are active, e.g., intermittently. Optionally, however, the output of the sound signal 19 can be silenced via the app.

When the exit time is over, the second relay 32 (the sound signal 19) switches off and the first relay 31 (the visual signalling LED 17) flashes to indicate that sanitization is in progress.
Disabling of sanitization: the sanitizing source 3 is disabled via the app (the device 38) or the switch 39, or by intervention of the presence sensor 13 or of one of the other sensors 15.

Once the corresponding signal has been received, the control device 5 opens the third relay 33 and the first relay 31 to indicate that the sanitization is switched off.

This step measures the value of the current in the load (the sanitizing source 3) by means of the amperometric sensor 35:

1) if the measured current is lower than a threshold value preset in the control device 5, this means that the sanitizing source 3 (e.g., the violet light source) is switched on in a non-hazardous manner, or is switched off;

2) if the measured current is higher than the preset threshold value (the sanitizing source 3 is activated above the threshold value), the control device 5 sends a command for reducing or switching off the power of the sanitizing source 3 to the respective driver 23. The sanitizing source 3, following this command, goes back to the pre-sanitization state (for example, a state where the sanitizing source 3 is switched off or operates below the threshold value). If the measured current still does not fall below the threshold value (which is a safe value for the exposure of users), the fourth relay 34 intervenes by cutting off the power to the driver 23 of the sanitizing source 3.

The presence sensor 13 and the other sensors 15 define a safety chain, and the control device 5 is configured to give consent to the use of the sanitizing source 3 only if the safety chain is closed, that is, if all safety criteria are met.

If, then, during operation in the sanitizing mode (sanitizing source 3 on), the control device 5 detects an interruption of the safety chain, e.g., the presence of a person detected by the presence sensor 13 or the opening of a door detected by one of the sensors 15, the control device 5 stops the sanitization by switching off the sanitizing source (or reducing its power supply below the safety threshold value).

In the event of a malfunction of the wireless communication system (or, in any case, of the communication system, which can also be wired, between the control device 5 and the presence sensor 13 and the other sensors 15), the control device 5 is still able to ensure user safety, because the amperometric sensor 35 monitors the current flowing in the sanitizing source 3, and if the source is switched on and a malfunction is detected in the communication system, it disables the sanitizing source 3.

Additional safety features are the alerts sent during sanitization by the visual signalling LED 17 and the sound signal 19.

The following settings are available through the app:
activation of the safety chain operation test;
on/off setting of the sound signal;
self-calibration of the current threshold value in the sanitizing source;
setting of the exit time prior to the switching on of the sanitizing source;
setting of the automatic sanitization switching off time.

The control device 5 allows a shared line to be used safely for all operating modes: in fact, the control device 5 discerns the operating mode actually in progress on the basis of the absorption detected by the amperometric sensor 5: if the amperometric sensor 35 detects a current higher than the threshold value, the lighting system 1 operates in the sanitizing mode (for example, night mode); if it detects a current lower than the threshold value, the lighting system 1 operates in the daytime mode (without sanitization). In the event of an interruption of the safety chain, as described above, the control device 5 intervenes and switches off the sanitizing source or reduces its power supply below the safety threshold value.

Lastly, it is understood that the control device and method as described and illustrated herein can be subject to modifications and variations that do not depart from the scope of the accompanying claims.

1. A control device (5) for controlling a lighting system (1) with sanitizing function, comprising: a printed circuit board (10) connectable to at least one lamp (2) of the lighting system (1) provided with at least one sanitizing source (3); at least one presence sensor (13) connected to the board (10) by a communication system (37), for example a wireless communication system; an amperometric sensor (35) connected to the sanitizing source (3) to measure the current in the sanitizing source (3); wherein the board (10) is configured so as to disable the sanitizing source (3) or to reduce the power supply to the sanitizing source below a threshold value when the presence sensor (13) detects the presence of users; or when a malfunction is detected in the communication system (37) and the amperometric sensor (35) detects in the sanitizing source (3) a current greater than the threshold value.

2. The control device according to claim 1, comprising at least one visual signalling LED (17) and/or a sound signal (19); and wherein the board (10) is configured so as to activate the visual signalling LED (17) and/or the sound signal (19) when the sanitizing source (3) is active.

3. The control device according to claim 1, wherein the board (10) is configured so as to measure the current in the sanitizing source (3) by means of the amperometric sensor (35); and if the measured current is lower than the threshold value, the control device (5) does not operate on the sanitizing source (3); if the measured current is higher than the threshold value, the control device (5) sends a command for reducing or switching off the power of the sanitizing source (3) to bring the sanitizing source (3) back to a pre-sanitization operation mode; and if the measured current still remains higher than the threshold value, the control device cuts off the power to the sanitizing source (3).

4. The control device according to claim 1, wherein the board (10) comprises: a supply input (11) for connection to a power supply; a sensor input (12) connected to the presence sensor (13); an outer sensors input (14) for connection to further sensors (15); a current measure input (24) for measuring the current in the sanitizing source (3); a transformer input (25) for connection to an amperometric transformer (26) for measuring the primary current in a driver (23) of the sanitizing source (3).

5. The control device according to claim 1, wherein the board (10) comprises: a first relay contact output (16) for connection to the visual signalling LED (17); a second relay contact output (18) for connection to the sound signal (19); a third relay contact output (20) for sanitization in progress; a fourth relay contact output (22) for connection in series to a driver (23) of the sanitizing source (3).

6. The control device according to claim 1, wherein the communication system (37) is a wireless communication system communicating with a remote device (38), for example a smartphone or another similar device, on which a dedicated application is loaded providing a user interface with the control device (5).

7. The control device according to claim 1, wherein the board (10) is configured so as to provide the consent to switch on the sanitizing source (3), following a switch-on command sent by a user via a remote device (38) or a switch (39), only if no users are detected by the presence sensor (13) and/or by the other sensors (15).

8. The control device according to claim 7, wherein the board (10) is configured to provide the consent to switch on the sanitizing source (3) after a preset exit time.

9. A lighting system comprising at least one light source emitting substantially white visible light, at least one violet light source with sanitizing capability, and at least one UV source; and a control device connected to said sources; characterized in that the control device is a control device according to claim 1.

10. A control method for controlling a lighting system (1) with sanitizing function having at least one lamp (2) provided with at least one sanitizing source (3); the control method comprising the steps of: detecting the presence of users in the environment in which the lighting system (1) is installed by means of at least one presence sensor (13) connected via a communication system (37), for example a wireless communication system, to a control device (5); monitoring by means of an amperometric sensor (35) connected to the sanitizing source (3) the current in the sanitizing source (3); disabling the sanitizing source (3) or reducing the power supply to the sanitizing source below a threshold value when the presence sensor (13) detects the presence of users; or when a malfunction is detected in the communication system (37) and the amperometric sensor (35) detects in the sanitizing source (3) a current greater than the threshold value.

11. The control method according to claim 10, comprising the steps of emitting a luminous and/or sound alarm when the sanitizing source (3) is active.

12. The control method according to claim 10, comprising the steps of: measuring the current in the sanitizing source (3) by means of the amperometric sensor (35); if the measured current is lower than the threshold value, the operation of the sanitizing source (3) is not changed; if the measured current is higher than the threshold value, a command for reducing or switching off the power of the sanitizing source (3) is sent to bring the sanitizing source (3) back to a pre-sanitization operation mode; and if the measured current still remains higher than the threshold value, the power to the sanitizing source (3) is cut off.

13. The control method according to claim 10, wherein the control device (5) provides the consent to switch on the sanitizing source (3), following a switch-on command sent by a user via a remote device (38) or a switch (39), only if no users are detected by the presence sensor (13) and/or by the other sensors (15).

14. The control method according to claim 13, wherein the consent to switch on the sanitizing source (3) is provided after a preset exit time.
